(19) United States
(12) Patent Application Publication
(10) Pub. No.: US 2008/0142453 A1
(43) Pub. Date: Jun. 19, 2008

(54) Biocidal composition and method for treating recirculating water systems

(76) Inventors: Michael Joseph Unhoch, Tyrone, GA (US); Sungmee Choi, Cheshire, CT (US); Katherine P. Roberts, Derby, CT (US); Leon Peter O'Malley, Hengoed (GB)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/999,392

(22) Filed: Dec. 5, 2007

Related U.S. Application Data
(60) Provisional application No. 60/874,573, filed on Dec. 13, 2006.

(51) Int. Cl. C02F 1/76; A61K 31/155; A01P 1/00

(52) U.S. Cl. 210/755; 514/635

(57) ABSTRACT

The present invention is directed to a composition for treating recirculating water systems, comprising: (1) a biocidal effective amount of a first nonoxidizing biocide comprising biguanide; and (2) a biocidal effective amount of a second nonoxidizing biocide comprising dibromonitrilopropionamide (DBNPA); wherein the composition is substantially free from oxidants. The present invention is also directed to a method of controlling the growth of microorganisms in recirculating water systems, comprising the step of treating the recirculating water systems with the above composition.

### CROSS-REFERENCE TO RELATED APPLICATIONS

This application claims the benefit of U.S. Provisional Patent Application Ser. No. 60/874,573 filed Dec. 13, 2006.

### BACKGROUND OF THE INVENTION

1. Field of the Invention

The present invention relates to treatment of water, and more specifically to treatment of water containing biofilm.

2. Description of the Related Art

Growth of microorganisms on surfaces exposed to water frequently result in the formation of biofilm. Biofilms are a collection of microorganisms surrounded by the slime they secrete, attached to either an inert or living surface. Biofilms are usually found on solid substrates submerged in or exposed to some aqueous solution, although they can form as floating mats on liquid surfaces. Given sufficient resources for growth, a biofilm will quickly grow to be macroscopic. Many problems result from development of biofilm on surfaces that are in contact with water.

Biofilm has been observed to accumulate in swimming pool and spa recirculation systems, even when sufficient sanitizer concentrations are maintained in the pool and spa water. This biofilm accumulation can lead to high consumption of the sanitizer and/or oxidizer used to maintain pool and spa water hygiene and clarity. It can also lead to severe filter blockage resulting in reduced circulation, channeling of the filter sand, failure of cartridges and diatomaceous earth grids leading to cloudy water. When the biofilm reaches a critical level it can slough off of the plumbing in sheets and also effect water clarity.

Biofilm formation (fouling) is also problematic in industrial water settings, for example in water recirculation systems, heat exchangers, cooling systems, pulp and paper processing, and the like. Biofilms can develop on the interiors of pipes, which can lead to clogging and corrosion. In extreme cases, biofilm may accumulate into large masses known as slime, and can interfere with valves, flowmeters, and other control equipment. Slime buildup can also reduce heat exchange or cooling efficiency on heat exchange surfaces.

Attempts have been made to control the growth of biofilm using chemical and physical treatments. Antimicrobial agents (generically termed biocides) have been used to eliminate, inhibit, or reduce biofilm proliferation. Typical biocides for this purpose include oxidizing biocides, such as ozone, chlorine dioxide, chlorine, iodine, and hydrogen peroxide, as well as non-oxidizing biocides, such as quaternary ammonium compounds, formaldehyde, and anionic and non-ionic surface-active agents. Examples of treatments for biofilm include the following:

U.S. Pat. No. 4,297,224 discloses use of 1-bromo-3-chloro-5,5-dimethylhydantoin as a treatment to control formation of biofilm in recirculating water.

U.S. Pat. No. 4,604,405 discloses use of synergistic combinations of 2-bromo-2-bromomethylglutaronitrile and 2,2-dibromo-3-nitrilopropionamide for inhibiting microbial growth.

U.S. Pat. No. 5,284,844 discloses use of 3,5-dihalogeno-1,2,6-thiadiazin-4-ones as biocides in the protection of materials and in water systems.

U.S. Pat. No. 6,395,189 discloses a method for reducing biofilm coatings and similar organic deposits in water systems, and more particularly, to an amine-formaldehyde condensate, optionally blended with surfactants in order to provide a composition useful as a biodispersant in cooling water systems.

U.S. Pat. No. 6,380,174 discloses a slime-removing composition comprising polyhexamethylenebiguanidine phosphate and 2-bromo-2-nitro-1,3-propanediol.

U.S. Pat. No. 7,008,545 discloses synergistic biocidal mixtures of a nitrogenous compound activated with an oxidant, and non-oxidizing biocides. However, such a combination requires the use of an oxidizer to activate the nitrogenous compound, and in some applications, an oxidizer is not desirable.

There is needed in the art improved compositions that reduce, control, or eliminate the development of biofilm. This invention is believed to be an answer to that need.

### SUMMARY OF THE INVENTION

In one embodiment, the present invention is directed to a composition for treating recirculating water systems, comprising: (1) a biocidal effective amount of a first nonoxidizing biocide comprising biguanide; and (2) a biocidal effective amount of a second nonoxidizing biocide comprising dibromonitrilopropionamide (DBNPA); wherein the composition is substantially free from oxidants.

In another embodiment, the present invention is directed to a method of controlling the growth of microorganisms in recirculating water systems, comprising the step of treating the recirculating water systems with a composition comprising: (1) a biocidal effective amount of a first nonoxidizing biocide comprising biguanide; and (2) a biocidal effective amount of a second nonoxidizing biocide comprising dibromonitrilopropionamide (DBNPA); wherein the composition is substantially free from oxidants.

These and other embodiments will be understood from the following drawings and detailed description of the invention.

### BRIEF DESCRIPTION OF THE DRAWINGS

The invention will be more fully understood from the following detailed description taken in conjunction with the accompanying drawings in which:

FIG. 1 is a graph showing remedial treatments and corresponding decreases in the amount of viable bacteria;

FIG. 2 is another graph showing remedial treatments and corresponding decreases in the amount of viable bacteria;

FIG. 3 is a graph showing results of remedial pool treatment over time; and

FIG. 4 is another graph showing results of remedial pool treatment over time.

### DETAILED DESCRIPTION OF THE INVENTION

Biofilms have been observed to form and propagate throughout recirculating water systems including but not limited to swimming pools, spas, heat exchangers, cooling systems, cooling towers, and the like. Biofilm has been observed to accumulate in swimming pool and spa recirculation systems even when sufficient sanitizer concentrations are maintained in the pool and spa water. This biofilm accumulation can lead to high consumption of the sanitizer and/or oxidizer used to maintain pool and spa water hygiene and clarity. It can also lead to severe filter blockage resulting in reduced circulation, channeling of the filter sand, failure of cartridges and diatomaceous earth grids leading to cloudy water. When the biofilm reaches a critical level it can slough off of the plumbing in sheets and also effect water clarity.

It has been unexpectedly discovered that an oxidant-free combination of a first nonoxidizing biocide comprising biguanide and a second nonoxidizing biocide comprising dibromonitrilopropionamide (DBNPA) displays a synergistic effect at preventing the establishment of common bacteria and fungi in the recirculating water system or in a visible biofilm that use of either biocide alone could not achieve. Through regular applications of the composition of the invention, it has been demonstrated that the biofilm can be remediated and the growth significantly reduced so as not to compromise the circulation and filtration systems or negatively impact water clarity. In the case where the biofilm problem has become manifest, the treatment method of the invention can restore plumbing, filter operation and water clarity by cleaning a significant amount of the biofilm from the circulation and filtration systems.

As indicated above, in one embodiment, the invention is a composition for treating recirculating water systems, comprising (1) a biocidal effective amount of a first nonoxidizing biocide comprising biguanide and (2) a biocidal effective amount of a second nonoxidizing biocide comprising dibromonitrilopropionamide (DBNPA), wherein the composition is substantially free from oxidants. Each of these components is discussed in greater detail below.

The first component of the composition of the invention is a first nonoxidizing biocide that comprises a biguanide compound. Any biguanide compound (or mixture of biguanidide compounds) may be included in this first nonoxiding biocide, including polymeric biguanides such as polyhexamethylene biguanide (PHMB). The polymeric biguanide preferably contains at least two biguanide units of Formula (1):

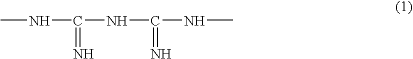

linked by a bridging group which contains at least one methylene (CH₂) group. The bridging group preferably includes a polymethylene chain, optionally incorporating or substituted by one or more hetero atoms such as oxygen, sulphur or nitrogen. The bridging group may also include one or more cyclic moieties which may be saturated or unsaturated. Preferably, the bridging group is such that there are at least three, and especially at least four, carbon atoms directly interposed between two adjacent biguanide units of Formula (1). Preferably, there are not greater than 10 and especially not greater than eight carbon atoms interposed between two adjacent biguanide units of Formula (1).

The polymeric biguanide may be terminated by any suitable group, such as a hydrocarbyl, substituted hydrocarbyl or by an amine group or by a cyanoguanidine group of the formula:

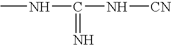

When the terminating group is hydrocarbyl, it is preferably alkyl, cycloalkyl, aryl or aralkyl. When the terminating group is substituted hydrocarbyl, the substituent may be any substituent which does not exhibit undesirable adverse effects on the microbiological properties of the polymeric biguanide. Preferred aryl groups include phenyl groups. Examples of suitable substituents are aryloxy, alkoxy, acyl, acyloxy, halogen and nitrile.

When the polymeric biguanide contains two biguanide groups of Formula (1), it is preferred that the two biguanide groups are linked through a polymethylene group, especially a hexamethylene group. The resulting structure can be termed a bisbiguanide.

The terminating groups in such a bisbiguanides are preferably C₁₋₁₀-alkyl groups which may be linear or branched, and optionally substituted aryl, especially optionally substituted phenyl. Examples of such terminating groups are 2-ethylhexyl and 4-chlorophenyl. Specific examples of such bisbiguanides are compounds represented by Formula (2) and (3) in the free base form:

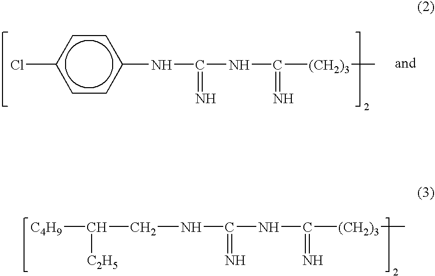

The polymeric biguanide preferably contains more than two biguanide units of Formula (1) and is preferably a linear polymeric biguanide which has a recurring polymeric chain represented by Formula (4) or a salt thereof:

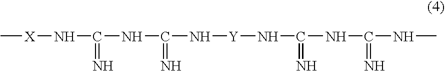

wherein X and Y represent bridging groups which may be the same or different and in which together the total of the number of carbon atoms directly interposed between the pairs of nitrogen atoms linked by X plus the number of carbon atoms directly interposed between the pairs of nitrogen atoms linked by Y is more than 9 and less than 17.

The bridging groups X and Y preferably consist of polymethylene chains, optionally interrupted by hetero atoms, for example, oxygen, sulphur or nitrogen. X and Y may also incorporate cyclic moieties which may be saturated or unsaturated, in which case the number of carbon atoms directly interposed between the pairs of nitrogen atoms linked by X and Y is taken as including that segment of the cyclic group, or groups, which is the shortest. Thus, the number of carbon atoms directly interposed between the nitrogen atoms in the group

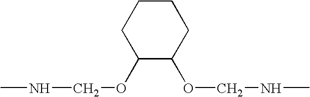

is 4 and not 8.

The linear polymeric biguanides having a recurring polymer unit of Formula (4) are typically obtained as mixtures of polymers in which they polymer chains are of different lengths. Preferably, the number of individual biguanide units of formulae:

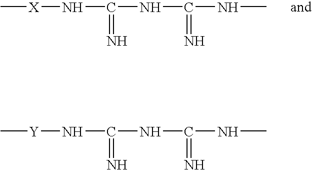

are, together, from 3 to about 80.

The preferred linear polymeric biguanide is a mixture of polymer chains in which X and Y are identical and the individual polymer chains, excluding the terminating groups, are of the Formula (5) or a salt thereof:

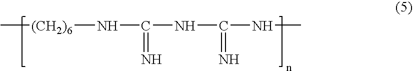

wherein n is from 4 to 40 and especially from 4 to 15. It is especially preferred that the average value of n is about 12. Preferably, the average molecular weight of the polymer is the free base form is from 1100 to 3300.

The linear polymeric biguanides may be prepared by the reaction of a bisdicyandiamide having the formula

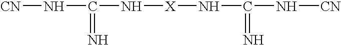

with a diamine H₂N—Y—NH₂ wherein X and Y have the meanings defined above, or by reaction between a diamine salt or dicyanimide having the formula

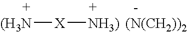

with a diamine H₂N—Y—NH₂ wherein X and Y have the meanings defined above. These methods of preparation are described in UK Patents 702,268 and 1,152,243, respectively, and any of the polymeric biguanides described therein may be used. These UK patents are incorporated by reference herein in their entireties.

As noted above, the polymer chains of the linear polymeric biguanides may be terminated either by an amino group or by a cyanoguanidine group:

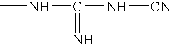

This cyanoguanidine group can hydrolyze during preparation of the linear polymeric biguanide yielding a guanidine end group. The terminating groups may be the same or different on each polymer chain.

A small proportion of a primary amine R—NH₂, where R represents an alkyl group containing from 1 to 18 carbon atoms, may be included with the diamine H₂N—Y—NH₂ in the preparation of polymeric biguanides as described above. The primary amine acts as a chain-terminating agent and consequently one or both ends of the polymeric biguanide polymer chains may be terminated by an —NHR group. These —NHR chain-terminated polymeric biguanides may also be used.

The polymeric biguanides readily form salts with both inorganic and organic acids. Preferred salts of the polymeric biguanide are water-soluable. When the polymeric biguanide is represented by a compound of Formula (2) in the free base form, a preferred water soluble salt is the digluconate. When the polymeric biguanide is represented by a compound of Formula (3) in the free base form, a preferred water soluble salt is the diacetate. When the polymeric biguanide is a mixture of linear polymers represented by Formula (5) in the free base form, the preferred salt is the hydrochloride.

It is especially preferred that the polymeric biguanide is a mixture of linear polymers, the individual polymer chains of which, excluding the terminating groups, are represented by Formula (5) in the hydrochloride salt form. This compound is commercially available from Arch Chemicals, Inc. (Norwalk, Conn.) under the trademark BAQUACIL.

The polymeric biguanide is preferably added to a recirculating water system to give a concentration thereof in the water of from 1 to 200 ppm, more preferably from 3 to 150 ppm, especially from 4 to 75 ppm, more especially from 6 to 20 ppm.

The amount of biguanide in the composition of the invention is any amount that results in a biocidal effect when added to a recirculating water system. In more specific embodiments, the amount of biguanide in the composition ranges from 0.1% to 40% by weight as liquid or 1% to 99% by weight as solid (in granular or compacted forms). Preferably 3% to 30% by weight as liquid or 10% to 75% by weight as a solid, more preferably 5 to 25% by weight as liquid or 15% to 60% by weight as a solid, most preferably 10 to 20% by weight as a liquid and 25% to 50% by weight as a solid, all weight percents being based on the total weight of the composition.

To result in a biocidal effect, the biocidal effective amount of biguanide in the composition preferably results in a final biocidal concentration in water of biguanide of between about 0.1 and about 500 ppm, more preferably between about 0.5 and 100 ppm, and most preferably between about 1 and 20 ppm.

The second component of the composition of the invention is a second nonoxidizing biocide that comprises dibromonitrilopropionamide (DBNPA). The amount of DBNPA in the composition of the invention is any amount that results in a biocidal effect when added to a recirculating water system. In more specific embodiments, the amount of DBNPA in the composition ranges from 0.1% to 40% by weight as liquid or 1% to 99% by weight as solid (in granular or compacted forms). Preferably 3% to 30% by weight as liquid or 10% to 75% by weight as a solid, more preferably 5 to 25% by weight as liquid or 15% to 60% by weight as a solid, most preferably 10 to 20% by weight as a liquid and 25% to 50% by weight as a solid, all weight percents being based on the total weight of the composition.

To result in a biocidal effect, the biocidal effective amount of DBNPA in the composition preferably results in a final biocidal concentration in water of biguanide of between about 0.05 and about 100 ppm, more preferably between about 0.1 and 50 ppm, and most preferably between about 0.25 and 25 ppm.

In addition, the most preferred concentration ratio of PHMB:DBNPA (as measured by PPM of the treated water) is 3:0.25 to 20:6 for preventing biofilm accumulation, and 1:1 to 1:3 for controlling existing biofilm.

The composition of the invention is substantially free from oxidants or oxidizing agents, such as chlorine, alkali and alkaline earth hypochlorite salts, hypochlorous acid, chlorinated isocyanurates, bromine, alkali and alkaline earth hypobromite salts, hypobromous acid, bromine chloride, halogenated hydantoin, ozone and peroxy compounds such as alkali and alkaline earth perborate salts, alkali and alkaline earth percarbonate salts, alkali and alkaline earth persulfate salts, hydrogen peroxide, percarboxylic acid, and peracetic acid. As defined herein, “substantially free” refers to less than 0.1% by weight.

The composition of the invention may also include one or more adjuvants selected from the group consisting of surfactants, biodisperants, biopenetrants, sorbitan monostearate, sulfamic acid, tallowpropylamine diamine, cocopropylamine diamine, oleylpropylamine diamine, stearyldimethylbenzylammonium chloride, DTEA II, and combinations thereof.

The amount of adjuvants that may be included in the composition of the invention ranges from 1% to 75% by weight as liquid or 5% to 75% by weight as solid (in granular or compacted forms). Preferably 5% to 70% by weight as liquid or 10% to 70% by weight as a solid, more preferably 10 to 60% by weight as liquid or 15% to 60% by weight as a solid, most preferably 20 to 50% by weight as a liquid and 25% to 50% by weight as a solid. Additionally, the most preferred concentration ratio of PHMB:DBNPA:Adjuvant (as measured in PPM in the treated water) is 10:0.5:10 to 20:3:50.

In use, the preferred amount of adjuvants in the composition of the invention preferably results in a final concentration in water of adjuvant of between about 5 and about 150 ppm, more preferably between about 15 and 100 ppm, and most preferably between about 20 and 50 ppm.

The composition of the invention may also contain additives known in the water treatment art. These additives include but are not limited to pigments, dyes, dissolution rate modifiers, binders, lubricants, color-containing salts, and the like. These additives may be pre-blended with either biocide component added to the mixture. Additionally, inert by-products such as water or lime may be present in the composition.

The making of the composition of the present invention can be accomplished by several different methods. For example, tumble blenders, v-blenders, ribbon blenders and the like may be used in a batch mode to blend the composition of the present invention. Additionally, screw augurs, conveyers, and the like may be used in a continuous mode to blend the composition. Such equipment and techniques are generally known in the art. Alternatively, the composition may be formed into a layered or homogeneously mixed solid shaped article. The composition of the present invention may be formed into a variety of solid shaped articles. These shaped articles include, but are not limited to tablets, bricks, briquettes, pellet, granules, and the like. The components of the present composition may also be melted, blended together, and then poured into a mold and allow the molten material to cool to room temperature. This production method also permits the composition to be made in one or more distinct layers. Another way of formulating the PHMB and DBNPA would be to melt one or more of the solids, blend them together, pouring them into a mold and allowing the molten material to cool to room temperature. This can also be accomplished as two distinct layers.

The composition and method of the present invention may be used in any recirculating water system where biofilm accumulates, for example swimming pools, spas, decorative ponds, as well as industrial applications, such as paper production plants, cooling towers, heat exchangers, waste water treatment, wood preservation applications, and the like. Significantly, the composition of the invention displays a synergistic effect between the two active ingredients which would not be predicted by one of skill in the art when considering each active ingredient individually.

In use as a treatment for swimming pools, the composition of the invention is added to a swimming pool recirculating water system to achieve the above concentration ranges and demonstrates a synergistic effect between the two biocides. The routine application (preventative application) of a combination of polyhexamethylene biguanide (1-20 ppm) and daily additions of dibromonitrilopropionamide (0.1-6 ppm), optionally with adjuvant, has shown a synergistic effect at preventing the establishment of common swimming pool bacteria and fungi in the water or in a visible biofilm that use of either biocide alone could not achieve. Remedial treatments that include a combination of polyhexamethylene biguanide (1-20 ppm) and additions of dibromonitrilopropionamide (3-24 ppm) with and without adjuvant also show a synergistic effect at remediating established populations of common swimming pool bacteria and fungi in the water and plumbing and filter that the use of either biocide alone could not achieve.

### EXAMPLES

### Example 1

### Remediation

Fungal isolates of common fungi found in swimming pools such as Paecilomyces lilacinus and Trichoderma spp. and bacterial isolates also common to swimming pools such as, i.e. Alcaligenes species and Sphingomonas species were used to construct a biofilm, which was treated with biocides, using the following method:

A mixture of fungal spore suspensions was prepared at a concentration of 3.65×10⁴/ml in 10% R2A broth. An aliquot of the spore suspension was added to each well of a 96-well plate, except column 12. The plates were incubated for 18 hours at 28° C. Also, a mixture of bacterial suspension was prepared at a concentration of 2.34×10⁵/ml in 20% R2A broth. An aliquot of bacterial suspension was added to each well of the 96-well plate, where fungal spores were placed. The plates were incubated for 24 hours at 28° C.

In a new 96-well plate, 6 ppm PHMB solution was added to each well, except column 1 and 12. A 100 ppm solution of biocide or adjuvant was added in the 6 ppm PHMB solution. An aliquot of the chemical solution was added to each well of column 1 and 2. The solution was mixed in each well of column 2 and an aliquot of solution was transferred from each well of column 2 to each well of column 3. The dilution was repeated to column 11 and the solution from each well of column 11 after dilution was discarded.

The culture broth was removed from each well of the incubated plate. An aliquot of 0.8% sodium chloride solution was added to each well, except column 12. The sodium chloride solution was then removed from each well. The prepared chemical solutions were transferred to the corresponding wells of the biofilm plate. The plate was incubated for 24 hours at 28° C.

The chemical solution was removed from the plate and an aliquot of resazurin solution was added to each well including column 12. The plate was kept on the lab bench for 24-48 hours. The plate was read at 540 nm and 620 nm. The difference in absorbance between the two wavelengths indicated the performance of the biocides. Table 1 illustrates efficacy of biocides tested.

TABLE 1
Effective Concentration with 6 ppm of PHMB of Biocides Tested

| Trade name | Chemical name | Efficacy level |
|---|---|---|
| DBNPA | Dibromonitrilopropionamide | >3 ppm |
| Uniquat QAC-50 | N-Alkyl (C12-16)-N,N- | >100 ppm |
| | dimethyl-N- | |
| | benzylammonium chloride | |
| Genapol C 050 | Fatty alcohol polyglycol | >100 ppm |
| | ether | |
| Alkamuls BR | Sorbitan monooleate | >100 ppm |
| Brij ® 72 | POE (2) stearyl ether | >100 ppm |
| Myristian | Mystic acid | >100 ppm |
| Armeen 12D | Dodecyl amines | >100 ppm |

As shown in Table 1, in the presence of 6 ppm PHMB, around 3 ppm of DBNPA was able to control all organisms within the biofilm matrix, whereas, the other candidates in Table 1 were shown to be ineffective.

Table 2. demonstrates the effect of different adjuvants on DBNPA efficacy against all organisms within the same biofilm matrix.

TABLE 2
Effective concentration of DBNPA in presence of PHMB and Adjuvant

| Biocide | ppm | Adjuvant | Description | ppm | Result |
|---|---|---|---|---|---|
| DBNPA | 3.13-6.25 | None | Na | Na | Na |
| DBNPA | 3.13-6.25 | Span 60 | Sorbitan | 50 | Similar to no |
| | | | monosterate | | adjuvant |
| DBNPA | 0.8-1.6 | Sulfamic acid | Organic acid | 50 | Most Significant |
| | | | | | improvement |
| DBNPA | 1.6-3.13 | Worm | Lonza Proprietary | 50 | Improvement |
| | | | blend of enzymes | | |
| | | | including lipase | | |
| DBNPA | 1.5-3.13 | SADA | Buckman | 50 | Improvement |
| | | | Proprietary | | |
| DBNPA | 1.6-3.13 | DSPO | Buckman | 50 | Improvement |
| | | | proprietary blend of | | |
| | | | organic penetrants | | |
| DBNPA | 6.25-12.5 | Envirosweet | 9,10 anthraquinone | 50 | Negative impact |
| DBNPA | 6.25-12.5 | Geogard 111A | Dehydroxyacetic | 50 | Negative impact |
| | | | acid (dehydroacetic | | |
| | | | acid) | | |
| DBNPA | 3.13-6.25 | DTEA | 2-(Decylthio)- | 50 | Similar to no |
| | | | ethylamine | | adjuvant |
| DBNPA | 3.13-6.25 | Triton BG10 | ethoxylated alkyl | 50 | Similar to no |
| | | | phenol | | adjuvant |
| DBNPA | 3.13-6.25 | Surfonic L24- | ethoxylated lauryl | 50 | Similar to no |
| | | 22 | alcohol | | adjuvant |

Table 2. shows that the sulfamic acid had the most significant effect on lowering the concentration of DBNPA needed to control all of the organisms in the biofilm and the Worm (proprietary blend of enzymes), SADA and DSPO also lowered the DBNPA concentration. None of the other adjuvants had positive impact on DBNPA's ability to control the organisms in the biofilm at lower concentrations.

### Example 2

### Prevention—Secondary Screen

The secondary screen methodology is based upon a laboratory scale model of a swimming pool. A volume (800 ml) of synthetic swimming pool water (Calcium chloride dihydrate and Sodium hydrogen carbonate solution) is pumped through a body of swimming pool filter sand, by means of a peristaltic pump. The water temperature of each system is maintained in the range of 80-90° F. The purpose of this experiment is to evaluate the robustness or the ability of the single or combinations of the biocides to prevent the microorganisms from establishing colonies in the water and sand filter media, thus preventing the formation of biofilm in the system. The performance of the biocide candidates were determined by the number of days the water clarity was maintained below 1.0 NTU, as well as, the number of bacterial and fungal counts upon exceeding this turbidity reading. It has been demonstrated that when the turbidity exceeds 1.0 NTU there are significant bacterial and fungal populations present in both water and sand. Also, a visible biofilm was observed in the sand and tubing when turbidity exceeds 1.0 NTU.

To the water, chemicals may be added as required. Typically, the concentration of Polyhexamethylene biguanide (PHMB) is maintained in the range of 0-10 ppm active ingredient, by addition of daily or weekly doses of the biocide. Hydrogen peroxide is added at concentrations of 0-27.5 ppm at the start of the experimentation, and the loss of hydrogen peroxide in each system was monitored by colorimetric assay. 2,2-Dibromo-3-nitrilopropionamide (DBNPA) is added either daily or weekly at a concentration range between 0-6 ppm.

The secondary model system is challenged on a daily basis with eight species of bacteria and four species of fungi typically found in swimming pool water. These microorganisms include species of the fungi Paecilomyces and Trichoderma, and species of the bacteria Alcaligenes, Chryseobacterium and Sphingomonas. Each inoculation represents a total addition of 0.8×10⁶ microorganisms per model apparatus.

A volume (5 ml) of synthetic bather load is added to the system on a daily basis, as a nutrient source for the microorganisms present in the system. The bather load consists of carbon, nitrogen and macro/micro nutrient sources such as urea, albumin, creatinine, lactic acid, uric acid, glucuronic acid, sodium chloride, sodium sulfate, ammonium chloride, sodium bicarbonate, potassium phosphate potassium sulfate.

The total number of viable bacteria and fungi present in each secondary model system is determined weekly, by conducting agar plate counts. Water samples are removed from each apparatus, and using this sample a dilution series (in 10⁻¹ steps, down to a 10⁻⁵ of the original sample) is prepared. Subsequently, an aliquot of each dilution is spread onto dry Cystine Lactose Electrolyte Deficient agar plates (for enumeration of bacteria) and dry Sabaroud-Dextrose agar plates (for enumeration of fungi). Bacterial and fungal plates are incubated for 3 and 5 days respectively at 30° C., prior to enumeration of the number of viable organisms.

Model water turbidity is measured on a daily basis, by measurement of water sample nephelometric turbidity units (NTUs), using a Hach 2100P turbidimeter. NTU measurement is conducted according to the manufacturer's instructions. PHMB concentration measurements are conducted daily by calorimetric assay, by reaction with 0.024% (w/v) Eosin Y and 10% (w/v) Sodium acetate trihydrate solution and measurement of the resultant color formation at 540 nm. Firstly, a Beer's Law plot for PHMB is constructed using PHMB solutions of known concentration. The resultant plot is then used to determine the PHMB concentration in secondary model water samples. Hydrogen peroxide concentration was followed by using Lovibond hydrogen peroxide low range test tablets in conjunction with a Lovibond PC22 photometer, operated according to the manufacturer's instructions.

The performance of the biocide candidates is determined by the number of days the water clarity is maintained below 1.0 NTU. It has been demonstrated that when the turbidity exceeds 1.0 NTU there are significant bacterial and fungal populations present in both water and sand. Also, a visible biofilm will be observed in the sand and tubing.

The data provided in Tables 3 and 4 below demonstrates the synergistic performance between PHMB and DBNPA (when the latter is added daily) at preventing the water from becoming turbid and controlling the bacteria and fungi that are added to the system. Data from experiment one (Table 3) reveals the enhanced performance of a combination of PHMB and DBNPA (with the latter being added daily at a rate of 1 ppm) compared to systems operated on PHMB alone. The DBNPA weekly addition with PHMB did not perform as well as the PHMB only treatment, revealing that DBNPA must be added regularly to result in enhanced performance.

The synergism between the biocidal activity of PHMB and DBNPA is clearly demonstrated from data generated in experiment 2 (Table 4), whereby addition of DBNPA in the absence of PHMB (added daily at a rate of 1 ppm) resulted in failure of the system and development of turbid water significantly sooner than systems dosed with PHMB alone or combinations of PHMB and DBNPA.

The data presented in Tables 3 and 4 also reveal that without addition of DBNPA at concentrations of 0.5 ppm (daily addition) or above, water clarity cannot be maintained in the absence of a hydrogen peroxide residual in the water. Conversely, combinations of PHMB and DBNPA (the latter being added daily at concentration of 0.5 and 1 ppm) retained water clarity for 19 and 69 days after complete loss of hydrogen peroxide from the system. This data clearly demonstrates the enhanced biocidal effect of a PHMB and DBNPA combination in the absence of any oxidizing agent.

TABLE 3
Example 1 of Performance of PHMB, DBNPA and Combinations
at Controlling Bacterial and Fungal Growth

| | | Bacterial | Fungal | | Number |
|---|---|---|---|---|---|
| | Number of | Counts at | Counts at | Day at which | of Days of |
| | Days of | Clarity of | Clarity of | Complete Loss | Clarity below |
| | Clarity | 1.0 NTU or | 1.0 NTU | of Hydrogen | 1.0 NTU with |
| Biocide and | below 1.0 | above | or above | Peroxide was | no Peroxide |
| Concentration | NTU | (CFU/ml) | (CFU/ml) | Recorded | in System |
| PHMB (3 ppm) | 50 | 6.65 × 10⁷ | 2.0 × 10E2 | 55 | 0 |
| PHMB (6 ppm) | 55 | 7.15 × 10⁴ | 0 | 55 | 0 |
| PHMB (3 ppm) + | 136 | 5.4 × 10⁵ | 0 | 67 | 69 |
| DBNPA (1 ppm) | | | | | |
| Daily addition | | | | | |
| PHMB (3 ppm) + | 34 | 6.7 × 10⁶ | 0 | 35 | 0 |
| DBNPA (6 ppm) | | | | | |
| Weekly addition | | | | | |

TABLE 4
Example 2 of Performance of PHMB, DBNPA and Combinations
at Controlling Bacterial and Fungal Growth

| | | Bacterial | Fungal | | Number |
|---|---|---|---|---|---|
| | Number of | Counts at | Counts at | Day at which | of Days of |
| | Days of | Clarity of | Clarity of | Complete Loss | Clarity below |
| | Clarity | 1.0 NTU or | 1.0 NTU | of Hydrogen | 1.0 NTU with |
| Biocide and | below 1.0 | above | or above | Peroxide was | no Peroxide |
| Concentration | NTU | (CFU/ml) | (CFU/ml) | Recorded | in System |
| PHMB (3 ppm) | 22 | 3.6 × 10⁷ | 6.0 × 10² | 23 | 0 |
| DBNPA (1 ppm) - | <12 | 9.9 × 10⁷ | 1.2 × 10³ | n/a | n/a |
| Daily Additions | | | | | |
| DBNPA (0.5 ppm) - | 54 | 1.3 × 10⁸ | 2.0 × 10² | 35 | 19 |
| Daily Additions | | | | | |
| DBNPA (0.25 ppm) - | 47 | 3.0 × 10⁷ | 3.0 × 10² | 47 | 0 |
| Daily Additions | | | | | |
| Chlorine (1 ppm) | 23 | 6.4 × 10⁴ | 0 | n/a | n/a |

### Example 3

### Remedial—Secondary Screen

The secondary screen methodology is based upon a laboratory scale model of a swimming pool. A volume (800 ml) of synthetic swimming pool water (Calcium chloride dihydrate and Sodium hydrogen carbonate solution) is pumped through a body of swimming pool filter sand, by means of a peristaltic pump. The water temperature of each system is maintained in the range of 80-90° F. The systems are allowed to fail resulting in turbid water and heavy bacterial and fungal growth. This is to simulate a swimming pool that has been improperly maintained and has a problem. The purpose of this test is to evaluate the ability of a single biocide or combination of biocides with adjuvants for controlling the organisms present in both the water and the filter sand. Turbidity is not a key measure because the suspended solids are too fine for filtration so they would need to be removed with a separate chemical treatment such as flocculation.

To the water, chemicals may be added as required. Typically, the concentration of Polyhexamethylene biguanide (PHMB) is maintained in the range of 0-10 ppm active ingredient, by addition of daily or weekly doses of the biocide. Hydrogen peroxide is added at concentrations of 0-27.5 ppm at the start of the experimentation.

The secondary model system is challenged on a daily basis with eight species of bacteria and four species of fungi typically found in swimming pool water. These microorganisms include species of the fungi Paecilomyces and Trichoderma, and species of the bacteria Alcaligenes, Chryseobacterium and Sphingomonas. Each inoculation represents a total addition of 0.8×10⁶ microorganisms per model apparatus.

A volume (5 ml) of synthetic bather load is added to the system on a daily basis, as a nutrient source for the microorganisms present in the system. The bather load consists of carbon, nitrogen and macro/micro nutrient sources such as urea, albumin, creatinine, lactic acid, uric acid, glucuronic acid, sodium chloride, sodium sulfate, ammonium chloride, sodium bicarbonate, potassium phosphate potassium sulfate.

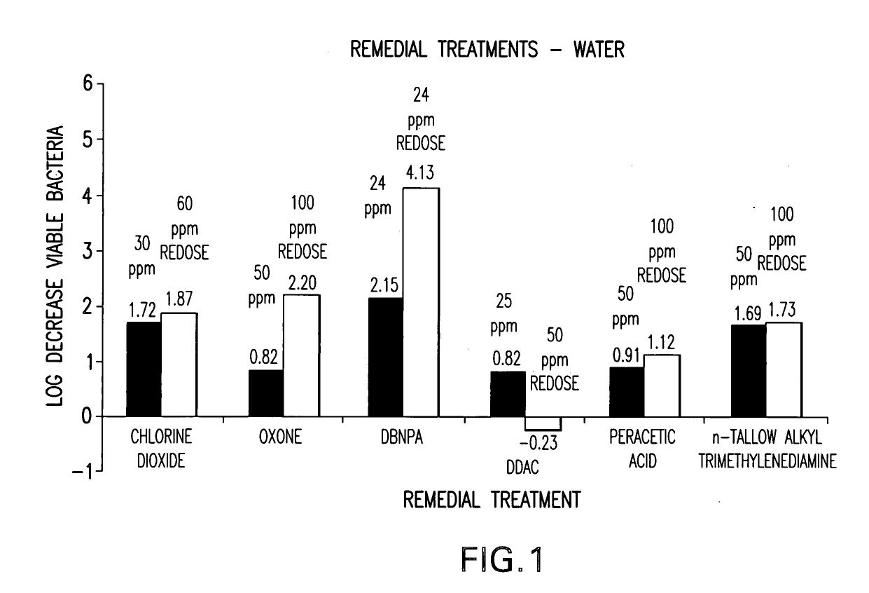

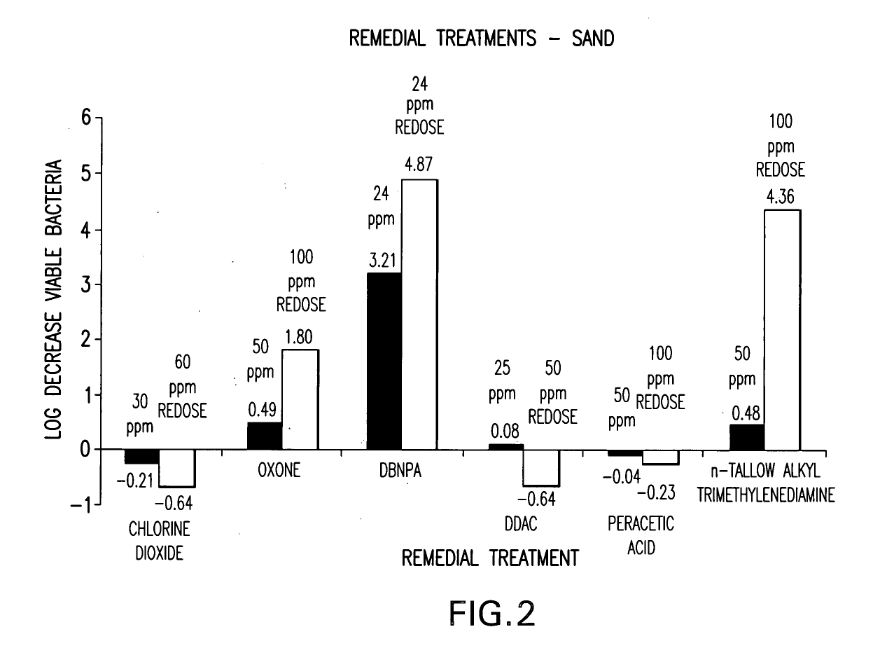

The systems are allowed to fail resulting in turbid water (>1.0 NTU) and heavy bacterial and fungal growth. The biocidal treatments are added and reduction of viable microorganisms is measured. The biocidal treatments may be added a second time. The performance is measured by the ability of the biocides or combinations to significantly reduce both viable bacterial and fungal populations. FIGS. 1 and 2 show how DBNPA performs significantly better than the oxidizers and surfactants.

FIGS. 1 and 2 illustrate the effectiveness of the oxidizing and non-oxidizing remedial treatment chemicals on pool water and sand that have significant bacterial and fungal counts as well as established biofilm on tubing and sand. They further show that the DBNPA gave similar log reductions of viable bacteria to the chlorine dioxide and oxone (potassium monopersulfate) and improved performance over the non-oxidizing surfactants of DDAC and n-tallow alkyltrimethylenediamine in the water. The performance of DBNPA was judged superior in log reduction of viable bacteria in the sand to both oxidizing and non-oxidizing candidates which was the more difficult media to treat because of the established biofilm.

### Example 4

### Prevention in Test Pools

The test pools were run in the preventative mode to evaluate the effect of the sanitizers on the build up of bacteria and fungi in the system. The pool pumps were operated for a minimum of 8 hours per day. Each pool was challenged twice per week with eight species of bacteria and four species of fungi typically found in swimming pool water. These microorganisms include species of the fungi Paecilomyces and Trichoderma, and species of the bacteria Alcaligenes, Chryseobacterium and Sphingomonas. Each inoculation represents a total addition of 0.8×106 microorganisms per test pool.

Synthetic bather load is added to the system on a weekly basis, as a nutrient source for the microorganisms present in the system. The bather load consists of carbon, nitrogen and macro/micro nutrient sources such as urea, albumin, creatinine, lactic acid, uric acid, glucuronic acid, sodium chloride, sodium sulfate, ammonium chloride, sodium bicarbonate, potassium phosphate potassium sulfate.

The total number of viable bacteria and fungi present in each test pool was determined weekly, by conducting agar plate counts. Water samples are removed from each test pool, and using this sample a dilution series (in 10-1 steps, down to a 10-5 of the original sample) is prepared. Subsequently, an aliquot of each dilution is spread onto dry Cystine Lactose Electrolyte Deficient agar plates (for enumeration of bacteria) and dry Sabaroud-Dextrose agar plates (for enumeration of fungi). Bacterial and fungal plates are incubated for 3 and 5 days respectively at 30° C., prior to enumeration of the number of viable organisms.

The control pools received a single dose of 27.5 ppm hydrogen peroxide and 3 ppm active PHMB (15 ppm as Sanitizer product) at the beginning of the study. Only the PHMB was maintained at 2-4 ppm active (10-15 ppm Sanitizer) by adding a single dose weekly.

The test pools for the first 135 day test period received an initial dose of 3 ppm active PHMB and daily doses of 0.5, 1.0 and 2.0 ppm active DBNPA. The PHMB was maintained at 2-4 ppm active (10-15 ppm Sanitizer) by adding a single dose weekly. There were no chemical doses administered on Saturday or Sunday.

On day 136 the PHMB was topped up in all of the test pools to 10 ppm active PHMB (50 ppm Sanitizer) and was maintained at 6-10 ppm active PHMB by adding a single weekly dose.

Pool 6 was also switched to biweekly tablet additions that delivered about 0.3 ppm active DBNPA on a daily basis and initial dose of 27.5 ppm hydrogen peroxide as well as weekly dosages of about 7 ppm.

Average water microbial count and count percentage data for the control and test pools are listed in Table 5 below.

TABLE 5
Average Water Microbial Count and Count Percentage Data

| | | Water | | | | | | |
|---|---|---|---|---|---|---|---|---|
| | | Mold | | | Bacteria | | | |
| | | Avg | %0 | % Counts >999 | Avg | %0 | % Counts >999 | |
| Pool # | Description | Counts | counts | cfu/ml | Counts | counts | cfu/ml | Comments |

Low Baquacil (10-20 ppm)

| | | | | | | | |
|---|---|---|---|---|---|---|---|
| 3 | Control | 2737 | 16.4 | 17.9 | 12,135 | 7.5 | 62.7 |
| 5 | Control | 4416 | 13.4 | 28.4 | 32,941 | 7.5 | 67.2 |
| 2 | 1.0 ppm/day | 4267 | 59.7 | 3.0 | 561 | 83.6 | 4.5 |
| | DBNPA | | | | | | |
| 6 | 0.5 ppm/day | 19.0 | 76.1 | 0.0 | 150 | 88 | 1.5 |
| | DBNPA | | | | | | |
| 7 | 2.0 ppm/day | 620 | 74.6 | 1.5 | 22.0 | 89.6 | 0.0 |
| | DBNPA | | | | | | |

Recommended Baquacil (30-50 ppm)

| | | | | | | | | |
|---|---|---|---|---|---|---|---|---|
| 5 | Control | 2900 | 28.6 | 9.5 | 2,700,000 | 9.5 | 73.8 | Two DBNPA Remedial |
| | | | | | | | | treatments were applied |
| | | | | | | | | during test |
| 2 | 1.0 ppm/day | 9.0 | 69.0 | 0.0 | 74.0 | 83.3 | 2.4 | |
| | DBNPA | | | | | | | |
| 6 | Tablet 0.3 | 8.0 | 66.7 | 0.0 | 5.0 | 92.8 | 0.0 | |
| | ppm/day DBNPA | | | | | | | |
| 7 | 2.0 ppm/day | 4.0 | 83.3 | 0.0 | 0.0 | 100 | 0.0 | |
| | DBNPA | | | | | | | |

A comparison of the average mold and bacterial counts and count frequencies of 0 (<10 cfu/ml) and >999 cfu/ml (denotes a treatment that is not maintaining good control) of control pools to test pools showed the following:

A. All pools treated with DBNPA exhibited significantly improved performance over controls by 2-4 logs.

B. Pool 2 at low PHMB had difficulty controlling mold compared to the other DBNPA pools. This may have been due to the location of Pool 2 because after a heavy rain because a large amount of dirt was observed which was not the case with any other pool. Mold could have entered with the soil which presented a greater challenge.

C. When the PHMB was increased to recommended ranges the average mold and bacterial counts in all pools treated with DBNPA were significantly reduced demonstrating synergy between PHMB and DBNPA. All of the average mold counts in the higher PHMB plus DBNPA treatments decreased by 3 logs to single digits vs. control pool and 1-2 log reduction vs. pools treated with lower concentrations of PHMB plus DBNPA.

### Example 5

### Remediation in Control Pools

The control pools were operated with below optimum sanitizer concentrations to encourage the build up of bacteria, fungi and possibly a mixed biofilm in the circulation and filtration system. The pool pumps were operated for a minimum of 8 hours per day. Each pool was challenged twice per week with eight species of bacteria and four species of fungi typically found in swimming pool water. These microorganisms include species of the fungi Paecilomyces and Trichoderma, and species of the bacteria Alcaligenes, Chryseobacterium and Sphingomonas. Each inoculation represents a total addition of 0.8×106 microorganisms per test pool.

Synthetic bather load is added to the system on a weekly basis, as a nutrient source for the microorganisms present in the system. The bather load consists of carbon, nitrogen and macro/micro nutrient sources such as urea, albumin, creatinine, lactic acid, uric acid, glucuronic acid, sodium chloride, sodium sulfate, ammonium chloride, sodium bicarbonate, potassium phosphate potassium sulfate.

The total number of viable bacteria and fungi present in each test pool was determined weekly, by conducting agar plate counts. Water samples are removed from each test pool, and using this sample a dilution series (in 10-1 steps, down to a 10-5 of the original sample) is prepared. Subsequently, an aliquot of each dilution is spread onto dry Cystine Lactose Electrolyte Deficient agar plates (for enumeration of bacteria) and dry Sabaroud-Dextrose agar plates (for enumeration of fungi). Bacterial and fungal plates are incubated for 3 and 5 days respectively at 30° C., prior to enumeration of the number of viable organisms.

The control pools received a single dose of 27.5 ppm hydrogen peroxide and 3 ppm active PHMB (15 ppm as Sanitizer product) at the beginning of the study. Only the PHMB was maintained at 2-4 ppm active (10-15 ppm Sanitizer) by adding a single dose weekly.

The control pools were remediated using the remedial treatment listed below when the turbidity exceeded 1.0 NTU and biofilm were visible in the skimmer and/or pool surfaces.

1. Brush surfaces with biofilm;

2. Add 6 ppm active DBNPA and 0.5 ppm and about 2 ppm polyaluminum chloride;

3. The water was circulated and pump turned off so the turbidity could settle;

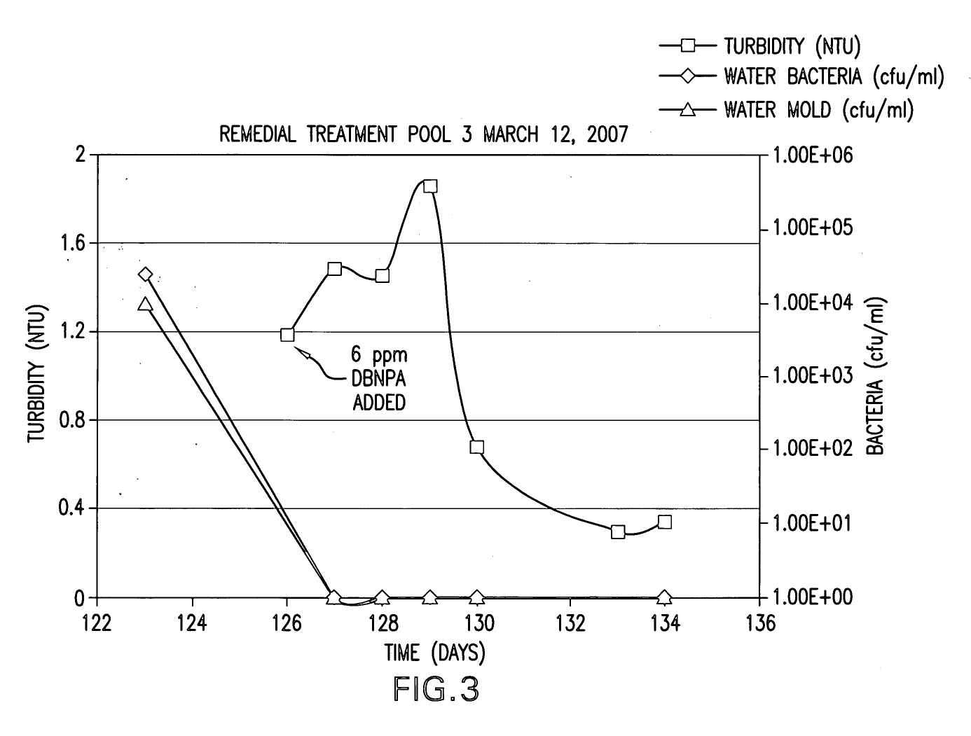

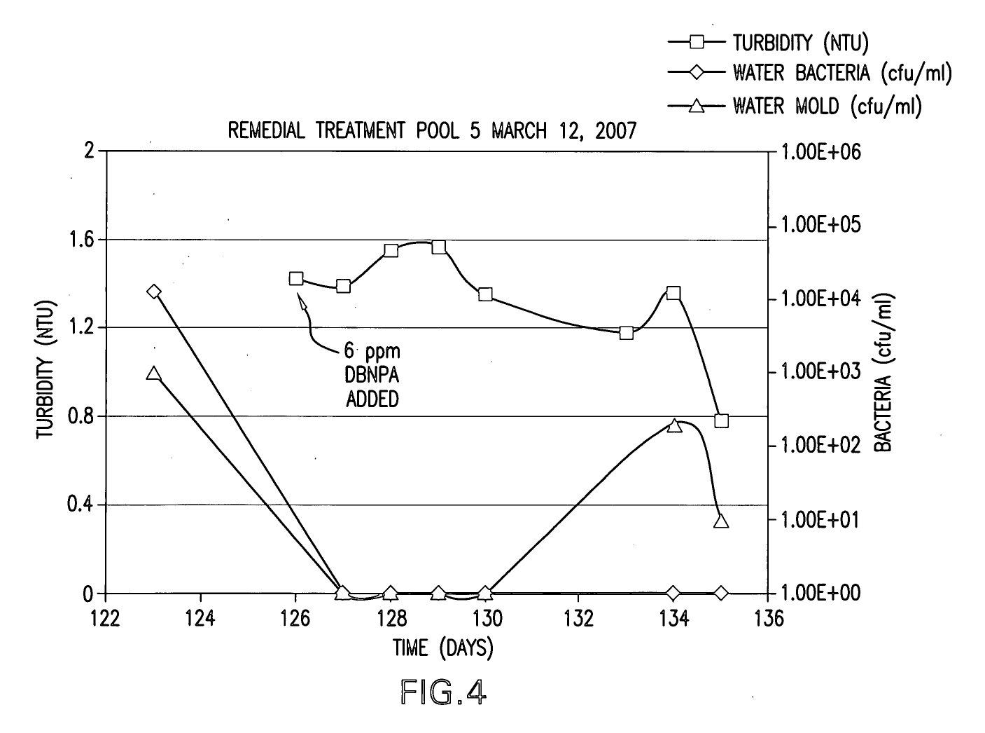

4. The pool was vacuumed when the floc settled. The antimicrobial and water quality results for two treatments are illustrated in FIGS. 3 and 4.

FIGS. 3 and 4 show that the remedial treatments in both pools gave an immediate 4 log reduction in bacterial counts in the water and 3 log reduction in fungal counts. The water clarity returned shortly thereafter. This data demonstrates the control of nuisance organisms that are difficult to control using PHMB alone.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A composition for treating recirculating water systems, comprising:
(1) a biocidal effective amount of a first nonoxidizing biocide comprising biguanide; and
(2) a biocidal effective amount of a second nonoxidizing biocide comprising dibromonitrilopropionamide (DBNPA);
wherein said composition is substantially free from oxidants.

2. The composition of claim 1, wherein said biguanide is polyhexamethylene biguanide (PHMB).

3. The composition of claim 1, wherein the amount of said biguanide in said composition ranges from about 0.1 to about 99% by weight, based on the total weight of said composition.

4. The composition of claim 1, wherein the amount of said biguanide in said composition ranges from about 5 to about 60% by weight, based on the total weight of said composition.

5. The composition of claim 1, wherein the amount of said biguanide in said composition ranges from about 10 to about 50% by weight, based on the total weight of said composition.

6. The composition of claim 1, wherein the amount of said biguanide in said composition results in a final biguanide concentration in water of between about 0.1 and about 500 ppm.

7. The composition of claim 1, wherein the amount of said biguanide in said composition results in a final biguanide concentration in water of between about 0.5 and about 100 ppm.

8. The composition of claim 1, wherein the amount of said biguanide in said composition results in a final biguanide concentration in water of between about 1 and about 20 ppm.

9. The composition of claim 1, wherein the amount of said DBNPA in said composition ranges from about 0.1 to about 99% by weight, based on the total weight of said composition.

10. The composition of claim 1, wherein the amount of said biguanide in said composition ranges from about 5 to about 60% by weight, based on the total weight of said composition.

11. The composition of claim 1, wherein the amount of said biguanide in said composition ranges from about 10 to about 60% by weight, based on the total weight of said composition.

12. The composition of claim 1, wherein the amount of said DBNPA in said composition results in a final DBNPA concentration in water of between about 0.05 and about 100 ppm.

13. The composition of claim 1, wherein the amount of said DBNPA in said composition results in a final DBNPA concentration in water of between about 0.1 and about 50 ppm.

14. The composition of claim 1, wherein the amount of said DBNPA in said composition results in a final DBNPA concentration in water of between about 0.25 and about 25 ppm.

15. The composition of claim 1, further comprising one or more adjuvants selected from the group consisting of surfactants, biodisperants, biopenetrants, sorbitan monostearate, sulfamic acid, tallowpropylamine diamine, cocopropylamine diamine, oleylpropylamine diamine, stearyldimethylbenzylammonium chloride, DTEA II, and combinations thereof.

16. The composition of claim 15, wherein the amount of said adjuvant in said composition ranges from about 1 to about 75% by weight, based on the total weight of said composition.

17. The composition of claim 15, wherein the amount of said adjuvant in said composition results in a final concentration of said adjuvant in water of between about 5 and 150 ppm.

18. A method of controlling the growth of microorganisms in a recirculating water system, comprising the step of treating said recirculating water system with a composition comprising:
(1) a biocidal effective amount of a first nonoxidizing biocide comprising biguanide, and wherein the final biguanide concentration in said recirculating water system is between about 0.1 and about 500 ppm;
(2) a biocidal effective amount of a second nonoxidizing biocide comprising dibromonitrilopropionamide (DBNPA), and wherein the final DBNPA concentration in said recirculating water system is between about 0.05 and about 100 ppm; and
(3) optionally, one or more adjuvants selected from the group consisting of surfactants, biodisperants, biopenetrants, sorbitan monostearate, sulfamic acid, tallowpropylamine diamine, cocopropylamine diamine, oleylpropylamine diamine, stearyldimethylbenzylammonium chloride, DTEA II, and combinations thereof, and wherein the final adjuvant concentration in said recirculating water system is between about 5 and 150 ppm;
and wherein said composition is substantially free from oxidants.

19. The method of claim 18, wherein the final biguanide concentration in said recirculating water system is between about 0.5 and about 100 ppm.

20. The method of claim 18, wherein the final biguanide concentration in said recirculating water system is between about 1 and about 20 ppm.

21. The method of claim 18, wherein the final DBNPA concentration in said recirculating water system is between about 0.1 and about 50 ppm.

22. The method of claim 18, wherein the final DBNPA concentration in said recirculating water system is between about 0.25 and about 25 ppm.
